(19) United States
(12) Patent Application Publication
(10) Pub. No.: US 2020/0023194 A1
(43) Pub. Date: Jan. 23, 2020

(54) Mid-infrared radiation health appliance

(71) Applicant: Thomas Skjoldborg Jensen, Vejle (DK)

(72) Inventor: Thomas Skjoldborg Jensen, Vejle (DK)

(21) Appl. No.: 16/483,772

(22) PCT Filed: Feb. 6, 2018
(86) PCT No.: PCT/EP2018/052905

§ 371 Date: Aug. 6, 2019

(30) Foreign Application Priority Data
Feb. 9, 2017 (DK) PA 2017 70086

(51) Int. Cl. A61N 5/06

(52) U.S. Cl. CPC A61N 5/0625; A61N 2005/0659; A61N 2005/0662; A61N 2005/0637

(57) ABSTRACT

A mid-infrared radiation health appliance includes i) a first portion, and a second portion, the first and second portions being arranged with a space in-between for a user; the first portion including a first cover plate having an upper surface facing the space adapted for supporting the weight of the user; ii) one or more elements for emitting infrared radiation and mounted in the first portion, iii) one or more elements for emitting infrared radiation and mounted in the second portion; iv) means for operating said elements at least by turning each element on and/or off. At least one or more elements of each portion emits infrared radiation at a wavelength of within 6-14 microns during use. The first cover plate is made of either a thermoplastic or plate glass material that transmit more than 50% of incident infrared radiation in the wavelength spectrum of 6-14 microns.

### TECHNICAL FIELD OF THE INVENTION

The present invention relates to the field of mid-infrared radiation health appliances.

### BACKGROUND OF THE INVENTION

Conventionally, there has been known a so-called far infrared radiation sauna for irradiating a whole body with far infrared radiation to promote blood circulation and perspiration. This far infrared circulation sauna is taken with the whole body put in a sealed housing, or only the head out. The whole body is heated up by far infrared radiation heating means arranged in the box.

In general, a conventional sauna has the following defects.

(1) The user is set on a chair arranged inside the housing when heated to elevated temperatures for forced perspiration. This unfavourably stimulates the blood circulation and muscles of the whole body while the body is strained;

(2) The sauna provides a temperature condition where the user's head is positioned at higher temperature than the feet; and

(3) The sauna is difficult to clean between sessions.

JP2007-136004 discloses a thermal medical treatment apparatus comprising heat radiation means structured in layers. The lower layer is a heat insulation material of e.g. polyurethane, the middle layer is heating element, and the upper layer is a 2 cm thick stone plate. The stone is natural noble serpentine. When the stone plate is heated, it emits infrared radiation. None of the emitted infrared radiation from the heating element is transmitted through the stone plate. Rather, the stone plate absorbs the infrared radiation and is heated. The stone plate thereby functions as a hot bed. This device is very expensive due to the stone plates, and the stone plates cannot withstand detergents making the device unsuitable for rental.

### OBJECT OF THE INVENTION

The objective of the present invention is to solve the above mentioned problems.

### DESCRIPTION OF THE INVENTION

A first aspect of the present invention relates to a mid-infrared radiation health appliance comprising:
a first portion (100), and a second portion (200), the first and second portions (100, 200) being arranged so as to define between them a space (300) for reception of a user; the first portion (100) comprises a first cover plate (120) comprising an upper surface facing the space (300) for reception of a user, which cover plate is adapted for supporting the weight of a user;
one or more elements (110) able to emit infrared radiation and mounted in said first portion (100),
one or more elements (210) able to emit infrared radiation and mounted in said second portion (200);
means for operating said elements (110, 210) at least by turning each element on and/or off;
wherein at least one or more elements (110, 210) of each portion (100, 200) emits infrared radiation at a wavelength of within 6-14 microns, such as within 7-13 microns, e.g. within 8-12 microns, such as within 9-11 microns, and preferably within 9-10 microns during use;
   the first cover plate (120) is made of either
a) a thermoplastic material that transmits more than 50%, such as within 50-99%, e.g. within 55-95%, such as within 60-90%, e.g. within 65-85%, such as within 70-80% of incident infrared radiation in the wavelength spectrum of 6-14 microns, or
b) a plate glass that transmits more than 50%, such as within 50-99%, e.g. within 55-95%, such as within 60-90%, e.g. within 65-85%, such as within 70-80% of incident infrared radiation in the wavelength spectrum of 6-14 microns.

A second aspect of the present invention relates to a mid-infrared radiation health appliance comprising:
a lower first portion, and an upper second portion, the portions being arranged so as to define between them a space for reception of a user;
one or more infrared emitting elements mounted in said first portion, one or more infrared emitting elements mounted in said second portion;
means for operating said infrared emitting elements, whereby a user can be subjected to infrared radiation when positioned in said space;
wherein the one or more infrared emitting elements are emitting infrared radiation at a wavelength of within 6-14 microns, such as within 7-13 microns, e.g. within 8-12 microns, such as within 9-11 microns, and preferably within 9-10 microns;
wherein the lower portion comprises a first cover plate adapted for supporting the weight of the user, and facing the space for the reception of a user; wherein the first cover plate is made of a thermoplastic material or a plate glass that that must transmit more than 50% of the infrared waves, such as within 50-99%, e.g. within 55-95%, such as within 60-90%, e.g. within 65-85%, such as within 70-80% at a frequency of within 6-14 microns, such as within 7-13 microns, e.g. within 8-12 microns, such as within 9-11 microns, and preferably within 9-10 microns.

A third aspect relates to a mid-infrared radiation health appliance comprising:
a lower portion, and an upper portion, the portions being arranged so as to define between them a space for reception of a user;
one or more infrared emitting elements mounted in said first portion, one or more infrared emitting elements mounted in said second portion;
means for operating said infrared emitting elements, whereby a user can be subjected to infrared radiation when positioned in said space;
wherein the one or more infrared emitting elements are emitting infrared radiation at a wavelength of within 6-14 microns, such as within 7-13 microns, e.g. within 8-12 microns, such as within 9-11 microns, and preferably within 9-10 microns;
wherein the lower portion comprises a first cover plate adapted for supporting the weight of the user, and facing the space for the reception of a user; wherein the first cover plate is made of a) a thermoplastic material that must transmit more than 50%, such as within 50-99%, e.g. within 55-95%, such as within 60-90%, e.g. within 65-85%, such as within 70-80% of the infrared radiation emitted by the one or more infrared emitting elements, or b) a plate glass that must transmit more than 50%, such as within 50-99%, e.g. within 55-95%, such as within 60-90%, e.g. within 65-85%, such as within 70-80% of the infrared radiation emitted by the one or more infrared emitting elements.

That the first portion comprises a first cover plate having a first surface facing at least partly upward means that a user may rest on the at least partly upward facing surface. The at least partly upward facing surface may be flat and/or horizontal, but it need not be flat or horizontal. The upper surface of the cover plate or the surface facing at least partly upward may be curved and/or inclined in order for a user to rest comfortably on the surface or in order for the emitted infrared radiation to transmit better through the cover plate.

The thickness of a cover plate in a mid-infrared radiation health appliance according to the invention, may depend on the position of the cover plate. If the cover plate is comprised in the first portion, a user will rest on the upper surface of the cover plate during use and the cover plate should therefore be able to support the user without breaking or bending or deforming to such an extent that the cover plate may get in contact with or move or break the element(s) emitting infrared radiation. The minimum thickness of a cover plate of the first or lower portion will depend on the material which is chosen for the cover plate, but in general the thickness of a supporting cover plate is at least 2 mm, normally at least 5 mm or at least 8 mm or at least 12 mm. The minimum thickness of a non-supporting cover plate or non-supporting parts of a cover plate may be smaller, such as at least 1 mm, normally at least 2 mm or at least 5 mm or at least 8 mm or at least 12 mm.

The maximum thickness of both supporting and non-supporting cover plates will normally be determined by absorption of radiation in the material, and by weight and price of the material. Normally, the maximum thickness of supporting and non-supporting cover plates will be 35 mm, or 25 mm or 15 mm.

This configuration allows for a user to lie down on the first cover plate, and thereby be in a relaxed position.

The term “mid-infrared” (MIR) generally covers the spectral region from approximately 3-50 microns (3000-50000 nm) according to ISO 20473. The inventor of the present invention found that a narrower range (6-14 microns, such as within 7-13 microns, e.g. within 8-12 microns, such as within 9-11 microns, or e.g. preferably within 9-10 microns) of infrared radiation is suitable for therapeutic use, such as deep tissue treatment.

The foremost attribute defining a suitable cover plate in relation to the present invention is transmission of radiation. When radiation is incident on a medium, a part of the radiation is reflected, a part of the radiation is transmitted or travelling through the medium, and a part of the radiation is absorbed in the medium.

According to the present invention transmission is a measure of throughput and is given as a percentage of incident radiation for a wavelength spectrum. Materials are usually either opaque in the visible spectrum (the material exhibit nearly 0% transmission in the visible spectrum), while transparent in the infrared region (the material exhibit nearly 100% transmission in the infrared region), or vice versa. Infrared transmission is defined as the proportion of incident infrared radiation, which passes through an object such as a cover plate and reaches a thermal imager enabling a measurement.

A traditional cover plate from a tanning bed made of Poly(methylmethacrylate) (PMMA) cannot be used as cover plate of the present invention, as PMMA is opaque to infrared radiation. Hence, special polymeric compositions allowing transmission of infrared radiation having a wavelength of 6-14 microns are needed. A suitable thermoplastic material or a plate glass transmit more than 50%, such as within 50-99%, e.g. within 55-95%, such as within 60-90%, e.g. within 65-85%, such as within 70-80% of incident infrared radiation at a wavelength of within 6-14 microns, such as within 7-13 microns, e.g. within 8-12 microns, such as within 9-11 microns, and preferably within 9-10 microns. Suitable examples of such material may be polyethylene, or polypropylene.

A cover plate is needed for two reasons. First, the user should not be able to touch the elements emitting infrared radiation, but more importantly, the lower portion should be cleaned between sessions to remove sweat and bacteria.

In one or more embodiments, the lower first portion, and the upper second portion comprises means adapted for moving the two portions relative to one another, such that the volume of the space for the reception of a user may be varied. The means may be adapted to move the two portions laterally relative to one another. The means may also or alternatively be adapted to pivotally move the two portions relative to one another.

In one or more embodiments, the upper portion comprises a second cover plate facing the space for the reception of a user, and wherein the second cover plate is made of a thermoplastic material or a plate glass that transmit more than 50%, such as within 50-99%, e.g. within 55-95%, such as within 60-90%, e.g. within 65-85%, such as within 70-80% of incident infrared radiation at a wavelength of within 6-14 microns, such as within 7-13 microns, e.g. within 8-12 microns, such as within 9-11 microns, and preferably within 9-10 microns.

In one or more embodiments, the upper portion comprises a second cover plate facing the space for the reception of a user, and wherein the second cover plate is made of a) a thermoplastic material that must transmit more than 50%, such as within 50-99%, e.g. within 55-95%, such as within 60-90%, e.g. within 65-85%, such as within 70-80% of incident infrared radiation emitted by the one or more infrared emitting elements, or b) a plate glass that transmit more than 50%, such as within 50-99%, e.g. within 55-95%, such as within 60-90%, e.g. within 65-85%, such as within 70-80% of incident infrared radiation emitted by the one or more infrared emitting elements.

In one or more embodiments, the first and/or the second portion comprises means adapted for closing of the foot end of the space for the reception of the user. This configuration allows for a better temperature control within the space.

In one or more embodiments, one or more infrared emitting elements are mounted in the means adapted for closing of the foot end of the space for the reception of the user; and wherein one or more infrared emitting elements are emitting infrared radiation at a wavelength of within 6-14 microns, such as within 7-13 microns, e.g. within 8-12 microns, such as within 9-11 microns, and preferably within 9-10 microns.

In one or more embodiments, the means adapted for closing of the foot end of the space for the reception of the user comprises a third cover plate facing the space for the reception of a user, and wherein the third cover plate is made of a thermoplastic material or a plate glass that transmit more than 50%, such as within 50-99%, e.g. within 55-95%, such as within 60-90%, e.g. within 65-85%, such as within 70-80% of incident infrared radiation at a wavelength of within 6-14 microns, such as within 7-13 microns, e.g. within 8-12 microns, such as within 9-11 microns, and preferably within 9-10 microns.

In one or more embodiments, the means adapted for closing of the foot end of the space for the reception of the user comprises a third cover plate facing the space for the reception of a user, and wherein the third cover plate is made of a) a thermoplastic material that transmit more than 50%, such as within 50-99%, e.g. within 55-95%, such as within 60-90%, e.g. within 65-85%, such as within 70-80% of incident infrared radiation emitted by the one or more infrared emitting elements, or b) a plate glass that transmit more than 50%, such as within 50-99%, e.g. within 55-95%, such as within 60-90%, e.g. within 65-85%, such as within 70-80% of incident infrared radiation emitted by the one or more infrared emitting elements. In one or more embodiments, the first and/or the second portion comprises means adapted for closing of the head end of the space for the reception of the user. This configuration allows for a better temperature control within the space.

In one or more embodiments, one or more infrared emitting elements are mounted in the means adapted for closing of the head end of the space for the reception of the user; and wherein one or more infrared emitting elements are emitting infrared radiation at a wavelength of within 6-14 microns, such as within 7-13 microns, e.g. within 8-12 microns, such as within 9-11 microns, and preferably within 9-10 microns.

In one or more embodiments, the means adapted for closing of the head end of the space for the reception of the user comprises a fourth cover plate facing the space for the reception of a user, and wherein the fourth cover plate is made of a thermoplastic material or a plate glass that will not absorb incident infrared radiation at a wavelength of within 6-14 microns, such as within 7-13 microns, e.g. within 8-12 microns, such as within 9-11 microns, and preferably within 9-10 microns, or at least absorb less than 40%, or less than 35% or less than 30%, or less than 20% of the incident infrared radiation at the specified wavelength.

In one or more embodiments, the means adapted for closing of the head end of the space for the reception of the user comprises a fourth cover plate facing the space for the reception of a user, and wherein the fourth cover plate is made of a) a thermoplastic material that transmit more than 50%, such as within 50-99%, e.g. within 55-95%, such as within 60-90%, e.g. within 65-85%, such as within 70-80% of incident infrared radiation emitted by the one or more infrared emitting elements, or b) a plate glass that must transmit more than 50%, such as within 50-99%, e.g. within 55-95%, such as within 60-90%, e.g. within 65-85%, such as within 70-80% of incident infrared radiation emitted by the one or more infrared emitting elements In one or more embodiments, the mid-infrared radiation health appliance further comprises means adapted for regulating the temperature within the space adapted for reception of a user.

In one or more embodiments, the mid-infrared radiation health appliance further comprises means adapted for directing and emitting a light of a visible colour onto the head, neck, throat, and/or chest of a user positioned within the space for reception of a user; wherein the one or more colours are selected from colours suitable for use in chakra therapy. This configuration is especially good in combination with infrared therapy, as the body is more receptive to chakra therapy when relaxed. These means must be positioned such that they are not shielded by the cover plate, as light at this wavelength will be absorbed by said cover plate. When one or more elements are mounted in a portion such as the first, the second, the third or the fourth portion, this means that the portion comprises further components beside the elements. E.g. the portion may comprise a frame and/or a cover plate facing the space for reception of a user and/or means for turning the elements on and off and/or outer cover plates facing surroundings and preventing access to the element(s) and/or handle(s) if the portion is movable between two or more positions etc. When a portion is provided with a cover plate having a surface facing the space for reception of a user, the cover plate is placed between the element(s) and the space for reception of a user during use, preventing a user positioned in the space from getting in direct contact with the element(s).

In one or more embodiments, the one or more infrared emitting elements are imbedded in the respective cover plate. Alternatively, the one or more infrared emitting elements are placed in distance from the surface of the cover plate facing away from the space for reception of a user, e.g. in a maximum distance of 10 cm, normally in a distance less than 5 cm, or less than 1 cm. In one or more embodiments, the one or more infrared emitting plate elements comprises a carbon membrane. Suitable examples of carbon membranes may be found in US20110081135 and U.S. Pat. No. 6,549,809, hereby incorporated by reference. As a nonlimiting example, the infrared emitting plate element comprises electrodes on both ends, in contact with a carbon black layer. When the carbon black layer is energized through the electrodes, the entire carbon black layer heats up by resistance heating, thereby radiating far infrared rays. Alternatively, the infrared emitting plate element comprises a frame, a thin layer, a grounding circuit board, a cloth layer and a second layer. The thin layer includes a carbon membrane and a printed circuit board. Two wires are used to connect the carbon membrane with a control device. The grounding circuit board includes a grounding layer, a circuit board, and a wire. The wire is used to connect the grounding circuit board with the ground. The grounding layer of the grounding circuit board may comprise a temperature regulating device, which can regulate the temperature of the infrared emitting plate element. The second layer, which has a crisscross pattern, and a plurality of fixing devices, is used to fixedly hold the thin layer, grounding circuit board, and cloth layer in the frame.

### Definitions

When the expression “during use” is applied, this refers to a state of the appliance where one or more of the elements are turned on and the temperature inside the space for reception of a user has been raised or is raising compared to the surrounding temperature.

As used in the specification and the appended claims, the singular forms “a”, “an”, and “the” also include plural referents unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about”, it will be understood that the particular value forms another embodiment.

It should be noted that embodiments and features described in context of one of the aspects of the present invention also apply to the other aspects of the invention.

### BRIEF DESCRIPTION OF THE FIGURES

FIG. 1 is a perspective view of a mid-infrared radiation health appliance in accordance with various embodiments of the invention.

### REFERENCES

100 First portion
110 Infrared emitting element
120 First cover plate
200 Second portion
210 Infrared emitting element
220 Second cover plate
300 Space
400 Means adapted for closing of the foot end of the space
410 Infrared emitting element
420 Third cover plate
500 Means adapted for closing of the head end of the space
600 Means adapted for emitting a light of one or more visible colours and directing the light to a user

### DETAILED DESCRIPTION OF THE INVENTION

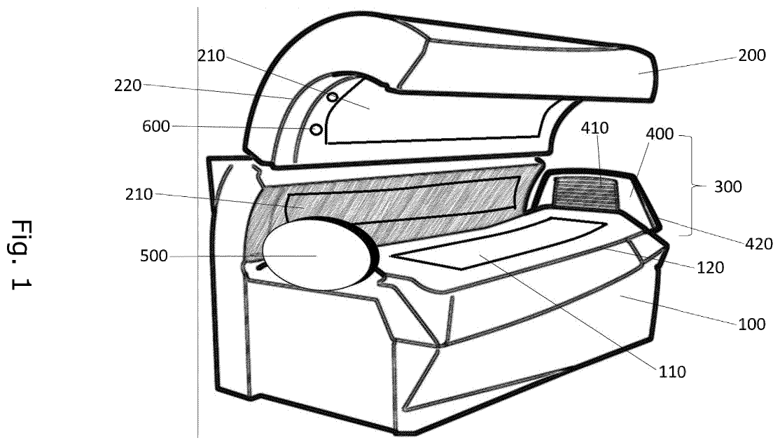

FIG. 1 is a perspective view of a mid-infrared radiation health appliance in accordance with various embodiments of the invention. The mid-infrared radiation health appliance comprises a lower first portion 100, and an upper second portion 200. The portions 100, 200 are arranged so as to define between them a space 300 for the reception of a user. One or more infrared emitting elements 110 are mounted in the first portion 100, and one or more infrared emitting elements 210 are mounted in the second portion. Means (not shown) for operating the infrared emitting elements 110, 210 are embedded in the portions 100, 200. The one or more infrared emitting elements 110, 210 are emitting infrared radiation at a wavelength of within 6-14 microns, such as within 7-13 microns, e.g. within 8-12 microns, such as within 9-11 microns, and preferably within 9-10 microns.

The lower portion 100 comprises a first cover plate 120 adapted for supporting the weight of the user and an upper surface of the first cover plate 120 faces the space 300 for the reception of a user.

The first cover plate 120 is made of a) a thermoplastic material able to transmit more than 50%, such as within 50-99%, e.g. within 55-95%, such as within 60-90%, e.g. within 65-85%, such as within 70-80% of incident infrared radiation which may be emitted by the one or more infrared emitting elements 110, or b) a plate glass able to transmit more than 50%, such as within 50-99%, e.g. within 55-95%, such as within 60-90%, e.g. within 65-85%, such as within 70-80% of incident infrared radiation which may be emitted by the one or more infrared emitting elements 110. Normally, the first cover material is made of a thermoplastic material such as polypropylene, as this material is low weight compared to plate glass, as well as scratch and chemical (such as cleaning agents) resistant.

The upper portion 200 comprises a second cover plate 220 facing the space 300 for reception of a user, and the second cover plate 220 is made of a) a thermoplastic material that is able to transmit more than 50%, such as within 50-99%, e.g. within 55-95%, such as within 60-90%, e.g. within 65-85%, such as within 70-80% of incident infrared radiation which may be emitted by the one or more infrared emitting elements 110, or b) a plate glass that is able to transmit more than 50%, such as within 50-99%, e.g. within 55-95%, such as within 60-90%, e.g. within 65-85%, such as within 70-80% of incident infrared radiation which may be emitted by the one or more infrared emitting elements 110. Normally, the second cover material is made of a thermoplastic material such as polypropylene, as this material is low weight compared to plate glass, as well as scratch and chemical (such as cleaning agents) resistant.

The first portion 100 comprises means 400 adapted for closing of the foot end of the space 300 for the reception of the user. One or more infrared emitting elements 410 are mounted in the means 400 adapted for closing of the foot end of the space 300 for reception of a user. The one or more infrared emitting elements 410 are emitting infrared radiation at a wavelength of within 6-14 microns, such as within 7-13 microns, e.g. within 8-12 microns, such as within 9-11 microns, and preferably within 9-10 microns. The means 400 adapted for closing of the foot end of the space 300 for the reception of the user comprises a third cover plate 420 provided with a surface facing the space 300 for reception of a user, and the third cover plate 420 is normally made of a) a thermoplastic material that is able to transmit more than 50%, such as within 50-99%, e.g. within 55-95%, such as within 60-90%, e.g. within 65-85%, such as within 70-80% of incident infrared radiation which may be emitted by the one or more infrared emitting elements 110, or b) a plate glass that is able to transmit more than 50%, such as within 50-99%, e.g. within 55-95%, such as within 60-90%, e.g. within 65-85%, such as within 70-80% of incident infrared radiation, which may be emitted by the one or more infrared emitting elements 110. Normally, the third cover plate material is made of a thermoplastic material such as polypropylene, as this material is low weight compared to plate glass, as well as scratch and chemical (such as cleaning agents) resistant.

The first portion 100 also comprises means 500 adapted for closing of the head end of the space 300 for reception of a user.

The lower first portion 100, and the upper second portion 200 comprises means (not shown, but their presence is indicated by the shape of the two portions) adapted for pivotally moving the two portions 100,200 relative to one another.

The mid-infrared radiation health appliance further comprises means (not shown) adapted for regulating the temperature within the space for the reception of the user e.g. this may include means for ventilation and cooling of the ventilation air and/or this may include control of current or voltage applied to one or more or all the elements emitting infrared radiation directed at least partly to the space adapted for a user. Current or voltage for each element may either be switched on and off, or the current or voltage may be gradually varied in order to control the temperature in the space during use.

The mid-infrared radiation health appliance also comprises means 600 adapted for directing and emitting a light of one or more visible colours onto e.g. head, neck, throat or chest or a combination of these body parts of a user positioned within the space 300 for reception of a user. The one or more colours are selected from colours suitable for use in chakra therapy such as Red (First chakra), Orange (Second chakra), Yellow (Third chakra), Green (Fourth chakra), Blue such as sapphire blue or turquoise (Fifth chakra), Purple or deep indigo (Sixth chakra) and/or White e.g. purplish white (Seventh chakra). The means 600 emit light having a wavelength between around 400 and 700 nm depending on the actual colour:
400-420 nm—wavelength of violet light
420-440 nm—wavelength of indigo light
440-500 nm—wavelength of blue light
500-520 nm—wavelength of cyan light
520-565 nm—wavelength of green light
565-590 nm—wavelength of yellow light
590-625 nm—wavelength of orange light
625-700 nm—wavelength of red light

1. A mid-infrared radiation health appliance comprising:
a first portion (100), and a second portion (200), the first and second portions (100, 200) arranged so as to define between them a space (300) for reception of a user;
one or more elements (110) able to emit infrared radiation and mounted in said first portion (100),
one or more elements (210) able to emit infrared radiation and mounted in said second portion (200);
wherein said one or more elements (110, 210) are operable by turning at least one of said one or more elements mounted in said first and second portions on or off;
wherein at least one element of said one or more elements (110, 210) of each portion (100, 200) emits infrared radiation at a wavelength of within 6-14 microns during use;
wherein the first portion (100) comprises a first cover plate (120) comprising an upper surface facing the space (300) for reception of a user, which cover plate is adapted for supporting the weight of a user; and wherein
the first cover plate (120) is made of either
a) a thermoplastic material that transmits more than 50% of incident infrared radiation in the wavelength spectrum of 6-14 microns, or
b) a plate glass that transmits more than 50% of incident infrared radiation in the wavelength spectrum of 6-14 microns.

2. The mid-infrared radiation health appliance according to claim 1, characterized in that the first cover plate (120) has a thickness of at least 2 mm, and a maximum thickness of 35 mm.

3. The mid-infrared radiation health appliance according to claim 1, characterized in that the second portion (200) comprises a second cover plate (220) facing the space (300) for reception of a user, and the second cover plate (220) is made of a) a thermoplastic material that is able to transmit more than 50% of incident infrared radiation, or b) a plate glass that is able to transmit more than 50% of incident infrared radiation in the wavelength spectrum of 6-14 microns.

4. The mid-infrared radiation health appliance according to claim 3, characterized in that the second cover plate (220) has a thickness of at least 2 mm, and a maximum thickness of 35 mm.

5. The mid-infrared radiation health appliance according to claim 3, characterized in that the first (120) and second (220) cover plates are made from polyethylene, polypropylene, or mixtures thereof.

6. The mid-infrared radiation health appliance according to claim 1, characterized in that the one or more elements (110, 210) able to emit infrared radiation are infrared emitting plate elements comprising a carbon membrane.

7. The mid-infrared radiation health appliance according to claim 3, characterized in that the first portion (100) or the second portion (200) comprises, or both the first portion and the second portion comprise means (400) adapted for at least partly closing of the foot end of the space (300) for reception of a user.

8. The mid-infrared radiation health appliance according to claim 7, characterized in that the first portion (100), or the second portion (200) comprises, or both comprise, means (400) adapted for at least partly closing of the foot end of the space (300) for reception of a user,
wherein one or more elements (410) emitting infrared radiation are mounted in the means (400) adapted for dosing of the foot end of the space (300) for reception of a user; and one or more of said elements (410) emits infrared radiation at a wavelength of within 6-14 microns during use, and wherein the means (400) adapted for closing of the foot end of the space (300) for reception of a user comprises a third cover plate (420) having a surface facing the space (300) for reception of a user, the third cover plate (420) is made of either a) a thermoplastic material that transmit more than 50% of incident infrared radiation in the wavelength spectrum of 6-14 microns, or b) a plate glass that transmit more than 50% of incident infrared radiation in the wavelength spectrum of 6-14 microns.

9. (cancelled)

10. The mid-infrared radiation health appliance according to claim 1, characterized in that the first portion (100), or the second portion (200) comprises, or both the first portion and the second portion comprise means (500) adapted for dosing of the head end of the space (300) for reception of a user, wherein one or more elements emitting infrared radiation are mounted in the means (500) adapted for closing of the head end of the space (300) for reception of a user; and one or more of these elements emit infrared radiation at a wavelength of within 6-14 microns during use, and wherein the means (500) adapted for closing of the head end of the space (300) for reception of a user comprises a fourth cover plate having a surface facing the space for reception of a user, the fourth cover plate is made of either a) a thermoplastic material that transmit more than 50% of incident infrared radiation in the wavelength spectrum of 6-14 microns, or b) a plate glass that transmit more than 50% of incident infrared radiation in the wavelength spectrum of 6-14 microns.

11-13. (canceled)

14. The mid-infrared radiation health appliance according to claim 1, characterized in that the appliance further comprises means adapted for regulating the temperature within the space (300) for reception of a user.

15. The mid-infrared radiation health appliance according to claim 10, characterized in that the appliance further comprises means (600) adapted for directing and emitting electromagnetic radiation onto the head, neck, throat, and/or chest of a user positioned within the space (300) for reception by a user; wherein the electromagnetic radiation comprises or is constituted of light of one or more visible colours having a wavelength of between 400 and 700 nm depending on actual colour, and the one or more visible colours are selected from a group of colours suitable for use in chakra therapy; wherein said means (600) are positioned such that they are not shielded by the first cover plate (120), the second cover plate (220), the third cover plate (420), or the fourth cover plate.
